(19) United States
(12) Patent Application Publication
(10) Pub. No.: US 2025/0371608 A1
(43) Pub. Date: Dec. 4, 2025

(54) A METHOD FOR GENERATING COMPRESSION GARMENT FIT INFORMATION AND AN APPARATUS THEREOF

(71) Applicant: Essity Hygiene and Health Aktiebolag, Goteborg (SE)

(72) Inventors: Peter Staab, Hamburg (DE); Sebastian Bannwarth, Buchholz in der Nordheide (DE); Michael Kisiel, Emmerich am Rhein (DE)

(21) Appl. No.: 18/851,890

(22) PCT Filed: Mar. 31, 2022
(86) PCT No.: PCT/EP2022/058717

§ 371 Date: Sep. 27, 2024

(51) Int. Cl. G06Q 30/0601

(52) U.S. Cl. CPC G06Q 30/06432

(57) ABSTRACT

Disclosed is a computer implemented method for generating compression garment fit information, the method including acquiring a video or images of a person; inputting the acquired video or images to an artificial intelligence module; determining the compression garment fit information by the artificial intelligence module; and outputting the compression garment fit information.

### CROSS REFERENCE TO RELATED APPLICATIONS

This application is a National Stage Application of PCT/EP2022/058717, filed Mar. 31, 2022, the contents of which are incorporated in their entirety herein.

### FIELD OF THE INVENTION

The present invention relates to a method for generating compression garment fit information and an apparatus thereof. More specifically, the present invention relates to a computer device and a computer implemented method for generating compression garment fit information. Furthermore, one or more artificial intelligence models are adopted in the apparatus and the method for generating the compression garment fit information.

### BACKGROUND ART

Garments which are able to apply pressure to a body part of a subject (e.g., a person, an animal, etc.) are known as compression garments and have been used for a variety of therapeutic and non-therapeutic applications, such as treating lymphedema, enhancing athletic performance or for cosmetic purposes. For example, the application of pressure to the affected (i.e., targeted) body part can alleviate symptoms of lymphatic disease and prevent or slow disease progression. Moreover, it may help in recovery after physical training.

Prerequisite for a successful compression therapy is a proper fit of the garment. A garment which fits poorly on the targeted body part will reduce its adherence to the patient, i.e., reduce the amount of time in which the patient is wearing the garment properly, and can neither elicit the desired compression level (e.g., deliver the expected compression force(s)) on all areas of the targeted body part. In particular for body parts which have a large diameter and/or an uneven surface morphology, it can be difficult to ensure a good, long-lasting fit of the garment. For such body parts, it has, thus, become routine to manufacture customized garments which are specifically adapted to the targeted body parts based on detailed measurements of the targeted body parts.

Proper measurements of the targeted body parts are, accordingly, a prerequisite for manufacturing such customized compression garments. Traditionally, these measurements can be taken manually by an experienced medical fitter/advisor. Studies have shown that the measurements taken by different fitters can differ quite considerably. The reliability of the measurements depends a lot on the experience of the fitter. During these measurements, the fitter is usually not (only) measuring circumferences of the affected body part in one or more locations on the skin surface (“Hautmaß”), but takes also so-called tension measurements (“Zugmaß”) where each of the circumferences is measured under tension, i.e., the measuring tape is pulled tight around the skin to apply a compression force on a location of the targeted body part. The purpose is to have a garment that is smaller/thinner than the targeted body part, e.g., a limb, so that an appropriate amount of compression force is exerted on the targeted body part.

When the customized garment is knitted or woven, the knitter (e.g., a machine or a person) may further change the dimensions to generate a compression gradient in the garment. For example, it is often advantageous or required that the garment has a higher compressive force in the lower calf area than in the upper calf area.

In order to make measurements more reliable and consistent, many attempts have been made to automate the generation of measurement data. For example, WO 2005/106087 A1 (University of Manchester) describes a method for making a pressure garment, including a step of defining 3D shape and pressure profile characteristics of a garment. The 3D shape and dimensions of the garment can be defined with the help of a 3D body scanner.

Several larger platforms are available that are able to take body measurements for production of customized compression garments. For example, the JOBST® LEXpert360 (BSN JOBST GmbH) is able to take a patient's skin circumference measurements. Other systems include the Bodytronic® 600 (Bauerfeind), the LegReader (Sigvaris) and the Rothballer 3D-ScanSystem (Rothballer Electronic Systems GmbH). These systems have relatively large hardware components, such as a platform on which the patient stands, one or more cameras that can circle around the patient and a computer and monitor that allows a real-time steering and assessment of the measurements. Accordingly, the systems are rather expensive and localized usually at an orthopedic shop which offers the service of fitting compression garments.

To make the systems less expensive and more flexible to use, mobile equipment has been suggested as a means for image acquisition and data processing. EP 3 435 800 B1 (LymphaTech, Inc.) describes a method for making compression garments in which digital images of the targeted body part are acquired by an operator moving an imaging device around the selected body part. The imaging device can be an iPad® or similar device to which a particular type of image acquisition device is attached, such as the Kinect 2® (Microsoft) (see of EP 3 435 800 B1). These approaches, thus, still make use of an advanced camera technology, such as structured light sensors or TOF technology. With the help of this technology, depth information is accumulated and subsequently used to generate 3D representations of the surface morphology. With these approaches it is still necessary to have dedicated equipment to carry out the scanning process and manufacture customized compression garments.

Therefore, there is a need of a method/apparatus which is easy to use but provides accurate compression garment fit information.

### SUMMARY OF THE INVENTION

The present invention relates to a method for generating compression garment fit information and an apparatus thereof. More specifically, the present invention relates to a computer device and a computer implemented method for generating compression garment fit information. Furthermore, one or more artificial intelligence models are adopted in the apparatus and the method for generating compression garment fit information.

A computer implemented method for generating compression garment fit information, the method includes: acquiring a video or images of a person; inputting the acquired video or images to an artificial intelligence module; determining the compression garment fit information by the artificial intelligence module; and outputting the compression garment fit information.

The artificial intelligence module may be a pretrained artificial intelligence model, such as a neural network model or a machine learning model.

The video or images may be 2D video and 2D images, respectively.

The artificial intelligence module may include a first artificial intelligence model, wherein the first artificial intelligence model may be configured to be pretrained to determine dimension information of the person.

The compression garment fit information may correspond to a body part of the person, and wherein the artificial intelligence module may include a second artificial intelligence model, the second artificial intelligence model being configured to be pretrained to determine the compression garment fit information corresponding to the body part of the person based on the determined dimension information.

The second artificial intelligence model may be configured to be pretrained to determine the compression garment fit information corresponding to the body part of the person further based on additional information of the person.

The compression garment fit information may include tension values.

Each of the tension values may be calculated based on a corresponding predetermined tension factor and corresponding skin surface dimension values.

The tension values may be determined by the second artificial intelligence model based on the determined dimension information and/or the additional information of the person, or the tension values may be determined by a third artificial intelligence model based on the determined dimension information and/or the additional information of the person.

The second artificial intelligence model may include different versions, each version being separately trained with data corresponding to a different targeted body part.

The second artificial intelligence model may include different versions, each version being separately trained with data corresponding to a different combination of a targeted body part and value ranges of parameters in the additional information.

The third artificial intelligence model may include different versions, each version being separately trained with data corresponding to a different targeted body part, and/or corresponding to a different combination of a targeted body part and value ranges of parameters in the additional information.

The second artificial intelligence model and/or the third artificial intelligence model may include a version being trained to target a body shape that is asymmetric.

The method may further include a step of receiving the additional information via user input.

The additional information may include at least one of height, age, weight, body mass index (BMI), and gender of the person.

The compression garment fit information may include at least one of the circumferences to be measured according to RAL-GZ387/1 or RAL-GZ387/2, for example a waist circumference, an upper hip circumference, a calf B1 circumference and a foot Y circumference.

The compression garment fit information may include fit information for one or more prefabricated and/or preconfigured compression garments.

A computing device is configured to perform the above method.

A storage medium is configured to store instructions configured to be executed by at least one processor to perform the above method.

### BRIEF DESCRIPTION OF THE DRAWINGS

The present invention will be discussed in more detail below, with reference to the attached drawings, in which:

FIG. 1 is a diagram showing a method for generating compression garment fit information.

FIG. 2 is a diagram showing a method for generating compression garment fit information.

FIG. 3 is a diagram showing some examples for compression garment measurement locations on the lower body part of a person according to RAL-GZ387.

FIG. 4 shows an example of a device.

FIG. 5 shows an example of logical modules within a device.

FIG. 6 shows the performance of the present invention.

### DESCRIPTION OF EMBODIMENTS

Embodiments of the present disclosure will be described herein below with reference to the accompanying drawings. However, the embodiments of the present disclosure are not limited to the specific embodiments and should be construed as including all modifications, changes, equivalent devices and methods, and/or alternative embodiments of the present disclosure.

The terms “have,” “may have,” “include,” and “may include” as used herein indicate the presence of corresponding features (for example, elements such as numerical values, functions, operations, or parts), and do not preclude the presence of additional features.

The terms “A or B,” “at least one of A or/and B,” or “one or more of A or/and B” as used herein include all possible combinations of items enumerated with them. For example, “A or B,” “at least one of A and B,” or “at least one of A or B” means (1) including at least one A, (2) including at least one B, or (3) including both at least one A and at least one B.

The terms such as “first” and “second” as used herein may modify various elements regardless of an order and/or importance of the corresponding elements, and do not limit the corresponding elements. These terms may be used for the purpose of distinguishing one element from another element. For example, a first element may be referred to as a second element without departing from the scope the present invention, and similarly, a second element may be referred to as a first element.

The expression “configured to (or set to)” as used herein may be used interchangeably with “suitable for,” “having the capacity to,” “designed to,” “adapted to,” “made to,” or “capable of” depending on the respective context. The term “configured to” does not necessarily mean “specifically designed to” on a hardware level. Instead, the expression “apparatus configured to . . . ” may mean that the apparatus is “capable of . . . ” along with other devices or parts in a certain context.

The terms used in describing the various embodiments of the present disclosure are for the purpose of describing particular embodiments and are not intended to limit the present disclosure. As used herein, the singular forms are intended to include the plural forms as well, unless the context clearly indicates otherwise. All the terms used herein including technical or scientific terms have the same meanings as those generally understood by an ordinary skilled person in the related art unless they are defined otherwise. The terms defined in a generally used dictionary should be interpreted as having the same or similar meanings as the contextual meanings of the relevant technology and should not be interpreted as having ideal or exaggerated meanings unless they are clearly defined herein. According to circumstances, even the terms defined in this disclosure should not be interpreted as excluding the embodiments of the present disclosure.

As disclosed in the background, it is very important to have accurate/reliable compression garment fit information (e.g., before the manufacturing a compression garment) in order for the compression garment to provide the desired effects. The fit information of a compression garment may provide an indication which size of a prefabricated and/or preconfigured, compression garment fits best. It is understood by a person skilled in the art that the preconfigured compression garments include prefabricated compression garments. Alternatively or additionally, the fit information may be or include dimension information for a customized garment. Accordingly, the method of the invention may be for generating compression garment fit information for selecting a prefabricated garment, dimension information for a customized garment, for manufacturing a customized compression garment, and/or any other relevant information compression garments.

When the fit information is for selection of a preconfigured or prefabricated compression garment, it may be one or more indicators on the fitting (i.e. matching) level(s) or ratio(s) between the targeted body part and one or more preconfigured or prefabricated compression garments, wherein the targeted body part is a body part that is going to wear the compression garment. The fitting (i.e., matching) level(s) or ratio(s) may be indicators indicating how well a preconfigured compression garment fits the targeted body part, e.g., via a predefined level (e.g., best fit “A”, good fit “B”, general fit “C”, loose fit “D”, not fit “E”, etc.), or via a ratio (e.g., a value between 0% and 100% where 0% indicating not fit at all, and 100% indicating a perfect fit), respectively. Some examples of fitting (matching) ratios may be found in EP 3 435 800 B1.

The body part may be any body part (i.e., any part of a body), in particular limb, amenable to compression therapy. For example, the body part can be the whole lower body, the whole upper body, a knee, waist, ankle, foot, arm or part of an arm (e.g., the upper arm and/or the lower arm) or a particular arm (e.g., left or right arm), thigh, leg or part of a leg (e.g. the lower leg and/or the upper leg) or a particular leg (e.g., left or right arm), etc., from a human body or an animal body. As another example, the body part may be a leg or a part of a leg, in particular the lower leg. In other examples, the body part may be an arm or a part of an arm, in particular a forearm.

The fit information may include information for the compression garments, e.g., at least one of one or more skin surface dimension values on one or more locations of the targeted body part, and/or one or more tension values on one or more locations of the targeted body part, and/or a decision on which of the preconfigured compression garments to be used (e.g., according to fitting/matching levels/ratios), and/or one or more manufacturing configurations (e.g., the sizes, the knitting/weaving method, material to be used, etc.) of the compression garment. The targeted body part of a compression garments may be distinguished between a left body part and the corresponding right body part (e.g., left/right leg and left/right arm), or may be a general body part without distinguishing the left and right side (e.g., leg and arm in general). Therefore, the fit information for a left body part and the corresponding right body part may be distinguished, or may not be distinguished.

The one or more skin surface dimension values may be dimension measurements of the targeted body part when there is no force on the body part, i.e., in its natural/relaxed state, e.g., at least one of circumferences, width, diameter, heights, and lengths on one or more locations. A “height” is measured in a straight line from one end of the respective body part (e.g., the sole of a foot for a leg dimension), while a length can also represent a dimension measured along a curved outline of a body part. Preferably, the skin surface dimension values (i.e., the group of skin surface dimension values included in the fit information) include one or more circumferences, e.g., 2 or more, 3 or more, 4 or more, or 5 or more. In other words, the skin surface dimension values can include from 1 to 26, from 3 to 26, or from 5 to 26 circumferences. These circumferences can be from different positions of the body part, e.g., as described elsewhere herein. Additionally, the skin surface dimension values can include one or more lengths of the respective body part, i.e., dimensions in longitudinal direction of the respective body part. Overall, the fit information that constitutes an output of the method of the invention can include e.g., from 1 to 40, from 10 to 40, from 15 to 40, from 20 to 40, from 25 to 40, from 30 to 40 different skin surface dimension values. The skin surface dimension values or the circumferences mentioned above can be values representing two different sides of the respective body part (e.g., the proper right and the proper left of the respective body part). The “proper left” is the side of the garment or body part that would be regarded by the wearer as the left side when the garment or body part is worn correctly. The “proper right” is the side of the garment or body part that would be regarded by the wearer as the right side when the garment is worn correctly. For example, if the body part is the lower half of the body, the skin surface dimension values or the can be a group of values representing the leg on the proper left and another group of values representing the leg on the proper right.

The one or more tension values of the targeted body part may be dimension values (i.e., measurements or dimension measurement values) of the targeted body part when there is a predetermined force on the body part (may be called tension dimension values), e.g., when a required force is applied to the body part in order to achieve the intended therapeutic or non-therapeutic effect. Historically, the tension values were acquired with a tape measure that was wrapped tightly around the respective body part, thereby reducing the circumference of the body part. The tension values may, hence, include circumferential dimension values. It has surprisingly been found that the artificial intelligence used in the method of the invention was able to generate very accurate tension values that can be used for garment selection or manufacture. The tension values as provided by a method of the invention can be calculated based on skin surface values of a circumference at a particular body part location and a tension factor. This is described in more detail elsewhere herein. The tension values are always smaller than the respective skin surface measurement of the circumference at the same body part location.

The decision e.g., on which of a number of preconfigured or prefabricated compression garments is to be used may be to determine the best fit compression garment for the targeted body part amongst a plurality of preconfigured compression garments.

The one or more manufacturing configurations of a corresponding compression garment may be the manufacturing parameters determining the compression garment sizes when producing the compression garments, e.g., including at least one of the weaving/knitting method, the material, the dimension configuration, etc.

The fit information may include dimension values (i.e., dimension measurements) such as circumference on a location of the targeted body part, length information, and an angle related to the body part (e.g., the max/min/normal angles between the upper and lower legs when a compression garment is for a knee). As described above, these dimension values may be skin surface dimension values and/or tension values.

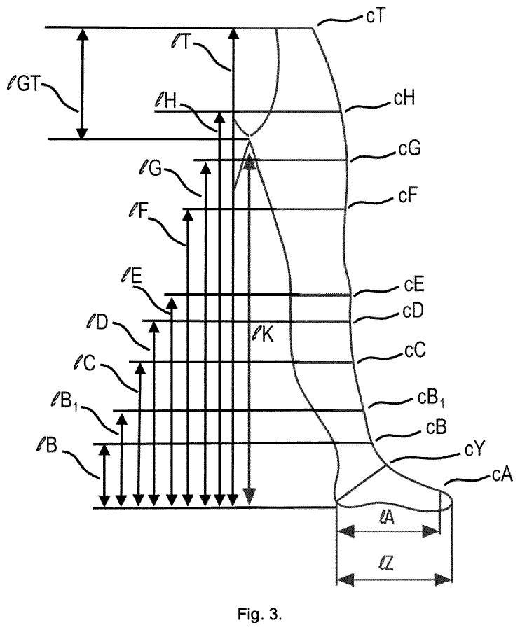

The locations of the dimension values (e.g., for skin surface dimension values and/or tension values) for compression garments may include any predetermined/predefined location on the targeted body part. Some examples of the predefined locations may be found in the standards of RAL-GZ387/1 and RAL-GZ387/2 for compression garments (for legs and arms, respectively) and in ISO8559 for general clothing. For example, the dimension values (e.g., circumferences and/or lengths/heights) may be obtained at multiple locations at the thigh, such as two or more circumferences from different heights of the tight, e.g. 3 or more, or 4 or more. For example, the dimension value locations for a human leg for compression garments are defined RAL-GZ387/1 as show in FIG. 3, where multiple circumference value locations (e.g., cT, cH, cG, cF, cE, cD, cC, cB1, cB, cY, cA), straight lengths (e.g., IT, IH, IK, LG, IF, IE, ID, IC, IB1, IB, IA, IZ, IGT) and other lengths (e.g., IKT) can be seen. For example, cT is the circumference of the waist, cH is the max circumference around the hip, cG is the circumference of the upper thigh, cF is the circumference of the lower thigh, cE is the circumference of the knee, cD is the circumference of the upper calf, cC is the max circumference of the calf, cB1 is the circumference of the lower calf, cB is the circumference of the ankle, CY is the circumference around the heel, and cA is the circumference of the foot. Furthermore, IT is the length between the waist and the heel, IH the length between the hip and the heel, IK is the length between the crotch and the heel, IG is the length between the upper thigh and the heel, IF is the length between the lower thigh and the heel, IE is the length between the knee and the heel, ID is the length between the upper calf and the heel, IC is the length between the calf and the heel, IB1 is the length between the lower calf and the heel, IC is the length between the ankle and the heel, IA is the length of the foot excluding the toes, IZ is the length of the foot and IGT is the length between the waist and the hip (until behind crotch). Other length may include the curve length over the skin surface from the front crotch and from the behind crotch (not shown in FIG. 3). Preferably, one or more of the dimension values provided by the method of the invention are circumference or length values being determined at the position(s)/location(s) defined in RAL-GZ387/1 and/or RAL-GZ387/2, such as 2 or more, 3 or more, 4 or more, 5 or more, 6 or more, 7 or more, 8 or more, 9 or more, 10 or more. In other words, from 1 to 26 of the dimension values provided by the method may be circumference of length values determined at the position(s)/location(s) defined in RAL-GZ387/1 and/or RAL-GZ387/2, e.g. from 5 to 26, from 10 to 26, from 15 to 26 or from 20 to 26.

Dimension values at one or more of these predefined locations may be included in the fit information. It is noted that the B1 location circumference is particularly important for compression garments. Having the value at B1 is a prerequisite for manufacturing compression stockings because it relevant to the Static Stiffness index, which is measured by putting a sensor between the compression stocking and the skin at the B1 location and measuring a difference in compression between the standing and lying position. It is therefore particularly valuable to include dimension values at the B1 position defined in RAL-GZ387/1.

As indicated above, the tension values may be dimension values (e.g., dimensional measurements) of the targeted body part when there is some tension or force applied to the targeted body part (e.g., in order to obtain desired effects from the compression garment). The tension values may be determined based on the skin surface dimension values, e.g., there is no need to apply actual tension/force on the targeted body part. For example, a tension factor, such as a reduction factor (e.g., 10% reduction, 15% reduction, etc.) may be applied to the skin surface dimension values in order to generate the tension values. The reduction factor may be different in different locations and/or different targeted body parts. The reduction factor may be different further depending on additional information, e.g., parameter of body mass index (BMI), height, type of disease (like diapedesis or lymphedema), age, height, gender, weight, etc. Each a combination of the parameters may correspond to a different reduction factor at each location of a particular body part. The reduction factor may be determined according to experiments or may be determined by a pretrained artificial intelligence model. Each combination of the parameters, the location and body part may be trained separately in sub artificial intelligence models, which can increase the accuracy of the tension factor prediction. The reduction factor may e.g. be a value selected from the range of from 0.50 to 0.99, e.g. from 0.60 to 0.95.

Alternatively, the tension values may be determined directly by an artificial intelligence model based on skin surface dimension values (and/or the additional information). For example, the model may be pretrained with skin surface dimension values (and/or the additional information) and correct tension values, such that after trained, the model can directly determine the tension values, i.e., without the intermediate decision on the tension factor.

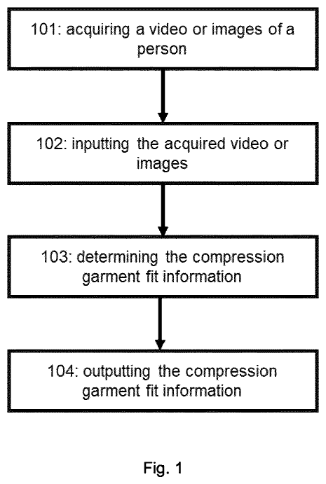

FIG. 1 is a diagram showing a method for generating compression garment fit information. Some of the steps in this method may be optional or combined, which a person skilled in the art would understand based on the present invention. For example, steps 101 and 104 may be optional, steps 101 and 102 may be combined.

In step 101, a video and/or images (later only referred to as “or” but including the option of “and”, or only referred to as “the video”) of a person may be acquired, from example, obtained via a device with a camera, or received by a device from an external device. The device/apparatus may be a general computing device, e.g., a laptop, a tablet, a mobile phone, a smart TV, a dedicated compression garment measuring device, etc. Preferably, the device is a tablet or a mobile phone. Surprisingly, it has been found that the 2D (two-dimension and/or two-dimensional) image acquisition that these devices provide for is sufficient to provide the accurate fit information that is required in the fitting and manufacture of compression garments. In other words, it is not necessary in the context of the invention to use camera equipment that has the ability to product 3D images, i.e., images that include depth information.

The video and/or images used in the context of the method of the invention may, accordingly, be 2D video and 2D images, respectively. This means that the video or images contain 2D information. Such 2D video or images do not contain depth information. For example, each of the pixels from the frames of the video or images can be represented on a 2D flat surface (e.g., by a two-dimensional vector including X and Y components) without depth information. Thus, the video/images are not 3D video/images, and no depth (i.e., height) information is acquired for their pixels in the present invention. Therefore, a normal camera may be able to capture/capture the required video/images in the present invention.

The acquired video or images may include one or more frames with the full body of the person. The acquired video or images may only include frames with the targeted body part for the compression garment. The video or images may include a full 360 degree view around the person (e.g., covering the front, left, back and right side of the body), from a distance, e.g., 2.5 meter.

In the acquired video or images, additional information may be acquired, for example the distance between the camera and the person (and/or the targeted body part) via a distance sensor (e.g., Lidar sensor or Ultrasonic sensor), the height of the person, the eye distance of the person, the distance between the ears of the person, angle information of the camera when taking the video/images, etc. Such distance and/or angle information may be used as a reference length/angle when determining the compression garment fit information, e.g., the length of the person, the waist circumference, etc. The acquisition of the additional information may be omitted. An additional item indicating a reference distance may be included in the video/images, e.g., the person may hold a coin or a credit card or they may be placed next to the person.

The acquired video or images may include multiple views of the person and/or the targeted body part, e.g., at least one of a front view, a back view, a left view, a right view, an up to bottom view with a certain angle and a bottom to up view with a certain angle, etc. These different views may provide a complete overview of the person or the targeted body part.

In step 102, the acquired video or images are input to determine the compression garment fit information in step 103, e.g., via an artificial intelligence module. The artificial intelligence module may be a part of the device acquiring the video or images, or in an external device, e.g., a remote server. The artificial intelligence module may include one or more artificial intelligence models, which are pretrained to determine compression garment fit information based on the input videos or images. The one or more artificial intelligence models may be neural network models, machine learning models, or any other artificial intelligence models.

The artificial intelligence module may include a single artificial intelligence model, which is pretrained to process the input video or images to directly output the garment fit information. The training data may include videos and images, and the corresponding garment fit information (e.g., measured by doctors or other experienced medical advisors, or directly the corresponding 3D models of the person/body part in the videos/images).

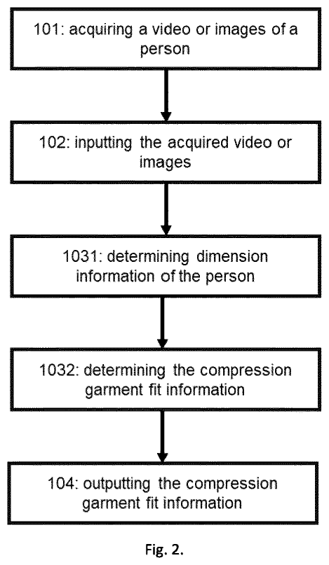

The artificial intelligence module may include multiple artificial intelligence models which divides in the determining of the fit information into multiple steps. For example, the artificial intelligence module may include two artificial intelligence models, where the first artificial intelligence model is configured to determine the skin surface dimension values without tension, and the second artificial intelligence model is configured to determine the tension values. Alternatively, the artificial intelligence module may include more artificial intelligence models, where a first artificial intelligence model is to determine the body is symmetric according to the key points or outline of the person in the video/images, a second artificial intelligence mode is to determine the skin surface dimension values based on whether the body is symmetric, a third artificial intelligence model is used to determine the tension values based on the skin surface dimension values and/or some additional information e.g., the types of the body part, the height, the weight, etc., and/or a fourth artificial intelligence model is used to determine the garment manufacturing configurations based on the tension values and/or the skin surface dimension values. Alternatively, the artificial intelligence module may include three artificial intelligence models, where a first intelligence model is to determine the skin surface dimension values, an optional second intelligence model is to determine the tension values based on the skin surface dimension values, a third intelligence model is to determine the fitting levels (and/or ratios) to preconfigured or prefabricated compression garments based on the skin surface dimension values and/or the tension values, wherein a selection of the preconfigured or prefabricated compression garments may be determined finally based on the fitting levels (and/or ratios). Thus, a person skilled in the art would understand based on the present document that there could be various of artificial intelligence models with the artificial intelligence module. FIG. 2 provides an example of the artificial intelligence module with two artificial intelligence models, which will be discussed in detail later.

In step 103, some additional information may be input (i.e., acquired) and used to determine the fit information, for example at least one of body mass index (BMI), height, type of disease (like diapedesis or lymphedema), age, height, gender, weight, etc. The additional information may be input by the user, received from an external device or prestored in the memory. The additional information provides reference information when determining the fit information, e.g., the height may provide a distance reference for the length and/or circumferences, the BMI and age may provide additional adjustments in the fit information, the gender (male or female) may provide certain corrections on different parts of the body, etc. Other additional information that may influence the fit information and known by a person skilled in the art may be considered in step 103 as well.

Furthermore, each combination of the additional information (i.e., combining different value ranges of the parameters in the additional information) may be trained in separate version of the artificial intelligence module, i.e., using dedicated/corresponding training data. For example, a first set of training data for female with a certain BMI range and age range is used to train the module and generate a first version of the module, and a second set of training data for male with the same BMI range and age range is used to train a copy of the same module to generate a second version of the module. Each of the versions may be selected according to the additional information when determining the fit information. Bodies of persons with various of diseases may also be trained separately in different versions of the artificial intelligence module. This separate training may generate more accurate fit information. Each combination of the additional information and a body part may be trained in separate versions of the artificial intelligence module as well. For example, a first set of training data for female left leg with a certain BMI range and age range is used to train the module and generate a first version of the module, and a second set of training data for male right leg with the same BMI range and different age range is used to train a copy of the same module to generate a second version of the module, etc.

Alternatively, only one version of the artificial intelligence module may be trained with all or a part of the additional information and the video/images as input by directly outputting the fit information.

In step 104, the garment fit information may be output and/or stored after it is determined. For example, it may be stored or displayed on a display of the device where the artificial intelligence module locates, and/or of the device where the camera is located. The garment fit information may also be output to an external device to be stored/displayed. And/or the garment fit information may be directly fed to a garment manufacturing machine.

FIG. 2 is a diagram showing a method for generating compression garment fit information. FIG. 2 is an example of FIG. 1 where step 103 is divided into two sub steps 1031, and 1032. Some of the steps in this method may be optional or combined, which a person skilled in the art would understand based on the present document, for example, steps 101 and 104 may be optional, steps 101 and 102 may be combined, and step 1031 may be optional.

Steps 101, 102 and 104 in FIG. 2 are the same as in FIG. 1, which will not be repeated here.

In step 1031, some general dimension (i.e., measurement) information of the person is determined. For example, some rough dimension values of the body of the person in the video/images such as one or more of the shoulder width, circumference of the chest, circumference of the underbust, circumference of the waist, circumference at the lower hips, circumference at the upper hips, circumference of the thigh, circumference of the ankle, length of the outseam, length of the inseam, length of arms, circumference of neck, circumference of calf, circumference of bicep, circumference of wrist, etc.

The general dimension information may include dimension information for one or two sides of the body, e.g., separate dimension values for the left leg and right leg, left arm and right arm, etc. The general dimension information may include dimension information for asymmetric body type. An asymmetric body type may be caused by various reasons, e.g., diseases like lymphedema, habits like using one of the arms/legs more at work, etc. The distinguishing of the dimension values for each left and right body part can provide accurate dimension values for asymmetric body type, which may additionally serve as input and can improve the performance of step 1032.

The rough/general dimension values may be directly output by a first artificial intelligence model included in the artificial intelligence module. Alternatively, the rough dimension values may be based on key points and/or silhouettes of the person in the video/images. For example, an artificial intelligence model may be trained to recognize the key points and/or silhouettes of the person in the video/images; and then another artificial intelligence model may be trained to determine the general dimension values based on the key points and/or silhouettes of the person. Optionally, the general dimension values may be prestored in the memory or received from an external device, e.g., these general dimension values may be measured manually by experienced fitter or based on a 3D model of the body or body part.

The first artificial intelligence model may be trained to directly determine the distinguishing of the dimension values for each left and right body part, e.g., determining the dimension values for the left body part and the corresponding right body parts individually. For example, the left arm and right arm are trained with separate data and after the model is trained, the model is able to determine the left arm and right arm dimension values separately.

An example of the first artificial intelligence model may be the models disclosed in WO2020239251A1, U.S. Pat. No. 10,706,262, or any other artificial intelligence models trained to achieve the same effect.

Optionally, before step 1031, there may be an additional step to first identify whether the body shape is an asymmetric body shape, e.g., based on the input videos and images. If it is determined that body shape is an asymmetric body shape, the output of the first artificial intelligence model can include separate dimension values for left body parts and the corresponding right body parts, and/or the first artificial intelligence model may input one or more indicators to indicate whether the body or body part is symmetric, of the asymmetric type (e.g., which body parts are asymmetric), etc. Otherwise, the artificial intelligence model may only output one set of values for symmetric body parts.

In step 1032, the compression garment fit information may be determined based on the general dimension information of the person. In this case, the garment fit dimension values may correspond to a targeted body part. A part or all of the general measurements may be used to determine the fit information for the targeted body part, such as, the left leg, the right arm, etc. Additional information of the person may be used as well when determining the fit information, for example, at least one of body mass index (BMI), height, type of disease (like diapedesis or lymphedema), age, gender, weight, etc.

In step 1032, a second artificial intelligence model included in the artificial intelligence module may be trained to determine the garment fit information, e.g., specific dimension values for the targeted body part. A compression garment is mostly only applied to a part of the body, i.e., a targeted body part (e.g. a stocking for a leg, an arm sleeve for an arm or a pantyhose for a lower body). Thus, detailed and accurate dimension values are necessary for the targeted body part.

The second artificial intelligence model may include a plurality of versions, e.g., each version can be pretrained with training data corresponding to a certain body part. For example, a first version may be trained to determine detailed fit information for the left leg or right leg, or legs in general (e.g., as shown in FIG. 3), and a second version may be trained to determine detailed fit information for the left arm or right arm, or arms in general. It is advantageous to have these two stages of determinations in steps 1031 and 1032, since the second artificial intelligence model is more focused and accurate on one body part without being disturbed by the noise from other body parts.

An optional third artificial intelligence model may be used to separately determine the tension values (i.e., dimension values under tension) and/or the tension factor(s), based on the output dimension values of the second artificial intelligence model (e.g., the skin surface dimension values for the targeted body part). Alternatively, the second artificial intelligence model may be directly trained to determine the tension values (e.g., the second artificial intelligence model includes multiple versions and each version corresponds to a particular targeted body part) or the tension factor(s) together with the other fit information, e.g., the skin surface dimension values, based on the general dimension information of the person from step 1301 and/or additional information.

An optional fourth artificial intelligence model may be included in the artificial module, which is trained to determine the fitting levels (or ratios) for preconfigured or prefabricated compression garments, e.g., based on at last one of the skin surface dimension values (e.g., output of the second artificial intelligence model), the tension values (e.g., output of the second artificial intelligence model or the third artificial intelligence model) and the general dimension information (e.g., output of the first artificial intelligence model). The fourth artificial intelligence model may in addition be trained to directly determine a selection of preconfigured or prefabricated compression garments, e.g., based on at last one of the skin surface dimension values (e.g., output of the second artificial intelligence model), the tension values (e.g., output of the second artificial intelligence model or the third artificial intelligence model) and the general dimension information (e.g., output of the first artificial intelligence model). Or an optional fifth artificial intelligence model is trained to directly determine the selection of preconfigured or prefabricated compression garments, e.g., based on at last one of the skin surface dimension values (e.g., output of the second artificial intelligence model), the tension values (e.g., output of the second artificial intelligence model or the third artificial intelligence model), the general dimension information (e.g., output of the first artificial intelligence model) and the fitting levels (or ratios) (e.g., output of the fourth artificial intelligence model).

In view of above for steps 1031 and 1032, the artificial intelligence module may include the first artificial intelligence models and at least one of the second to fifth models. For example, the artificial intelligence module may include the first and second artificial intelligence models, or the first, second and third artificial intelligence models, the first and third artificial intelligence models, or the first, second and fourth artificial intelligence models, or the first, second, third and fourth artificial intelligence models, the first, third and fourth artificial intelligence models, or the first, second and fifth artificial intelligence models, or the first, second, third and fifth artificial intelligence models, the first, third and fifth artificial intelligence models, or the first, second, fourth and fifth artificial intelligence models, or the first, second, third, fourth and fifth artificial intelligence models, the first, third, fourth and fifth artificial intelligence models, or any other combinations.

In each of the second to fifth artificial intelligence models, the additional information of the person may be used as well, for example at least one of body mass index (BMI), height, type of disease (like diapedesis or lymphedema), age, height, gender, weight, etc. The additional information may be input by the user, received from an external device or prestored in the memory. The additional information may provide reference information when determining the fit information, e.g., the height may provide a distance reference for the fit information, the BMI and age may provide additional adjustments in the fit information, the gender (male or female) may provide certain corrections for different body parts, etc. Other additional information that may influence the fit information and is known by a person skilled in the art may be considered in step 1032 as well.

Furthermore, each combination of the additional information (i.e., combining different value ranges of the parameters in the additional information) may be trained in separate versions of the second or the third artificial intelligence model. For example, a first set of training data for female with a certain BMI range and age range is used to train an artificial intelligence sub model and generate a first version of the artificial intelligence sub model, and a second set of training data for male in the same BMI range and age range is used to train the artificial intelligence sub model and generate a second version of the artificial intelligence sub model; different data sets corresponding to different diseases may also be used to train the sub models separately to generate different versions of the sub models. This separate training can generate more accurate fit information for each of the combined situations. Each combination of the additional information and a body part may be trained in separate versions of the artificial intelligence module as well. For example, a first set of training data for female left leg with a certain BMI range and age range is used to train the module and generate a first version of the module, and a second set of training data for male right leg with the same BMI range and different age range is used to train a copy of the same module to generate a second version of the module, etc.

Before step 1032, an additional step may be included in the method, i.e., a determination of whether the targeted body part is symmetric to the corresponding body part, for example, the targeted body part may be the left leg, and the corresponding body part is the right leg. The determination may be based on an output of step 1031 indicating whether the targeted body part is symmetric, or the determination may be via comparison of the output general dimension values of step 1031. For example, step 1031 may output the dimension values for each body part individually even for body parts which are normally symmetric; then the dimension value(s) for the targeted body part (e.g., the left leg) may be compared to the dimension value(s) for the corresponding body part (e.g., the right leg); if the differences of the corresponding dimension values are larger than certain thresholds, then it can be determined that the targeted body part is an asymmetric body part.

If the target body part is determined to be an asymmetric body part, a dedicated version or more versions of the second and/or third artificial intelligence model may be trained, particularly for the targeted body part when it is asymmetric. The versions may also take into account: the additional information, for example, disease information, occupation/type of job, age, weight, etc. This means that each combination of value ranges of the parameters in the additional information and the asymmetric body part has its own trained version.

After step 1032, one or more additional artificial intelligence models may be added. For example, a fourth artificial intelligence model may be pretrained and used to determine the manufacturing configuration of the compression garment based on the skin surface dimension values, tension values and/or the additional information of the person.

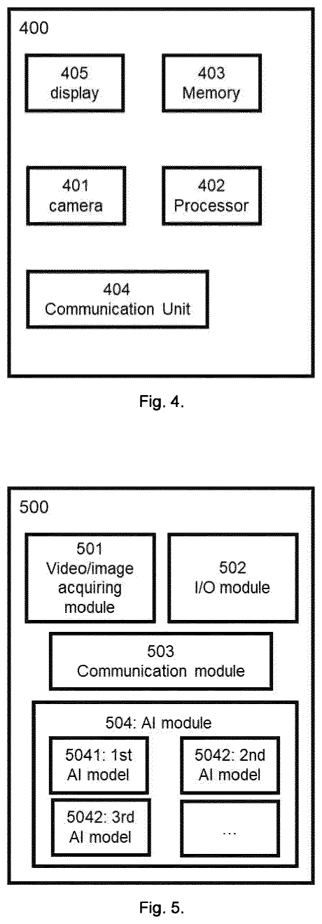

FIG. 4 shows an example of a device/apparatus. The device 400 may be a computer implemented device, which implements the methods in FIG. 1 and FIG. 2. Some obvious/optional components/units are omitted in FIG. 4. The device 400 may, however, additionally include such components and/or units which are obvious to a person skilled in the art, e.g., a separate input unit, a microphone, battery, etc.

The device 400 may include a camera 401, a processor 402, memory 403, a communication unit 404 and a display 405.

The camera 401 may be configured to capture the video or images in step 101 for the determination of the compression garment fit information. The camera is, in particular, adapted to capture 2D video or images. The aforementioned video or images are, hence, 2D video or images, respectively. The video or images captured by the camera 401 may be stored in the memory 403, or may be directly fed to the processor 402, which is configured to determine the garment fit information. Alternatively, the video or images may be prestored in the memory 403 or received via the communication unit 404 from an external device, and output to the processor 402. The memory 403 may be configured to store the artificial intelligence module (including one or more the artificial models) in the methods of FIG. 1 and FIG. 2. The models are executed by the processor 402 to determine the compression garment fit information as disclosed in FIG. 1 and FIG. 2. The compression garment fit information may be output via the display 405, or to an external device via the communication unit 404, which may also be stored in the memory 403. The display 405 may display the videos or images during and after the video/image capturing. The display 405 may further be an input device to receive input from a user, e.g., a touch screen. The received input may relate to the additional information used in any of the artificial intelligence module and models as disclosed in FIG. 1 and FIG. 2. The inputs may be received via a separate input device, which is not shown in FIG. 4.

FIG. 5 shows an example of logical modules within a device/apparatus, which is configured to perform the methods in FIG. 1 and FIG. 2. Some obvious modules are omitted in FIG. 5 for device 500, e.g., the power supply module, the audio/video processing module, internal communication module, etc.

The device 500 may include a video/image acquiring module, which is configured to perform step 101 in FIGS. 1 and 2 (e.g., from a camera or the memory). Alternatively, the video/images may be received via the communication module 503 from an external device. The input/output (I/O) module 502 may be configured to receive inputs from a user and to output information. The inputs may include the additional information of the person in the methods of FIGS. 1 and 2; and the output information may include the compression garment fit information and/or the video/images in the methods of FIGS. 1 and 2.

The device 500 may include an artificial intelligence (AI) module 504, which is configured to determine the compression garment fit information based on the captured video/images. The artificial intelligence module 504 may include one or more artificial intelligence models 5041, 5042, 5043, . . . 504N. When there is only one artificial intelligence model 5041, the artificial intelligence module 504 may be configured to be pretrained to determine the compression garment fit information directly from the video/images of the person and/or the additional information of the person. When there are more than one artificial intelligence models, each of the artificial intelligence models may be configured to be pretrained to determine some intermediate information (which may be used as input for other models). For example, the output of the first artificial intelligence model 5041 may be used as the input of the second and third artificial intelligence models 5042 and 5043; the output of the second artificial intelligence model 5042 may be used as the input of the third artificial intelligence model 5043; . . . ; until the last artificial intelligence model 504N to output the compression garment fit information.

For example, the first artificial intelligence model 5041 may be configured to determine the general dimension values of the person based on the video/images; the second artificial intelligence model 5042 may be configured to determine a first part of the compression garment fit information (e.g., skin surface dimension values) for the targeted body part based on the general dimension values and/or the additional information of the person; the third artificial intelligence model 5043 may be configured to determine a second part of the compression garment fit information (e.g., the tension values or tension factor(s)) based on at least one of the general dimension values, the additional information and the first part of the compression garment fit information. Each of the artificial intelligence models may be one of the artificial intelligence models disclosed in the methods FIGS. 1 and 2 and will not be repeated here.

The present invention may be a storage medium configured to store instructions to perform the methods in FIGS. 1 and 2. The instructions are configured to be executed by at least one processor.

Based on the present invention, accurate fit information can be obtained in an easy way from 2D (two-dimension and/or two-dimensional) video/images.

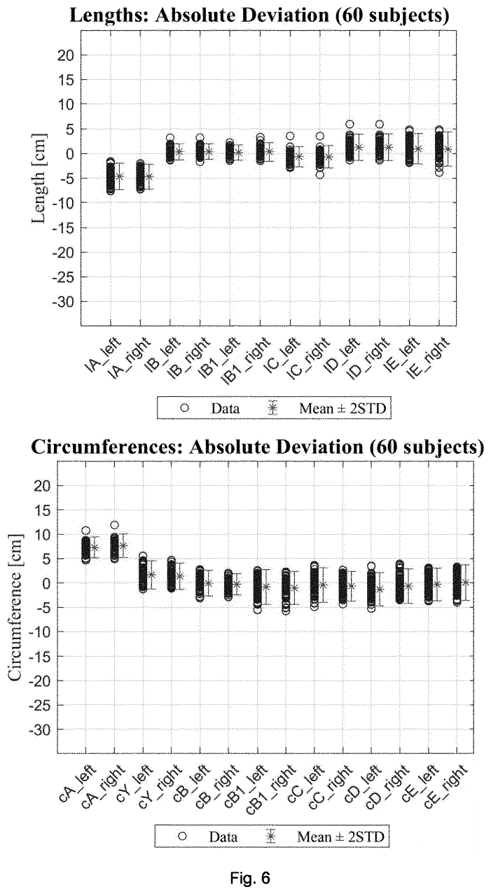

FIG. 6 shows the performance of the present invention, using 2D video or images, where the deviations between the actual circumference and length values of legs (e.g., based on the leg dimension value locations as shown in FIG. 3) and the output of the garment fit information by the present invention (e.g., the predicted/output skin surface dimension values from the videos/images of the legs) are shown. The “data” in FIG. 6 indicates the deviations between the predicted values and the actual values, and the left and right indicate whether the value is for the left leg or right leg. For example, if the data is “0”, it means that the predicted value is accurate (i.e., the predicted value is the same as the real value). It is hereby proven that the predicted values are surprisingly sufficiently accurate when compared to the real values. The minimal deviations shown in FIG. 6 demonstrate that the present invention can make much better predictions than the state of art solutions.

Another example of the performance of the present invention is shown in the table 1, wherein the tension factors based on real (manually acquired) data and based on the predicted data (i.e., the present invention) are compared. The real tension factor is the tension factor provided by experienced medical advisors, i.e., the tension factor that is used in real life. The predicted tension factor is the output of the present invention. It can be seen that the predicted tension factors are very close to the real tension factors and have very low standard deviations.

TABLE 1
Tension factors

| | cA | cY | cB | cB1 | cC | cD | cE | cF | cG |
|---|---|---|---|---|---|---|---|---|---|
| Real | 0.930 | 1.000 | 0.910 | 0.890 | 0.900 | 0.950 | 0.930 | 0.880 | 0.880 |
| tension | | | | | | | | | |
| factor | | | | | | | | | |
| Predicted | 0.963 | 1.026 | 0.914 | 0.893 | 0.904 | 0.951 | 0.928 | 0.838 | 0.880 |
| tension | | | | | | | | | |
| factor | | | | | | | | | |
| Standard | 0.024 | 0.023 | 0.011 | 0.024 | 0.010 | 0.020 | 0.019 | 0.040 | 0.019 |
| deviation | | | | | | | | | |
| of the | | | | | | | | | |
| Predicted | | | | | | | | | |
| tension | | | | | | | | | |
| factor | | | | | | | | | |

1. A computer implemented method for generating compression garment fit information, the method comprising:
acquiring a video or images of a person;
inputting the acquired video or images to an artificial intelligence module;
determining the compression garment fit information by the artificial intelligence module; and
outputting the compression garment fit information.

2. The method of claim 1, wherein the artificial intelligence module is a pretrained artificial intelligence model, such as a neural network model or a machine learning model.

3. The method of claim 1, wherein the video or images are 2D video and 2D images, respectively.

4. The method of claim 1, wherein the artificial intelligence module comprises a first artificial intelligence model, wherein the first artificial intelligence model is configured to be pretrained to determine dimension information of the person.

5. The method of claim 4, wherein the compression garment fit information corresponds to a body part of the person, and wherein the artificial intelligence module comprises a second artificial intelligence model, the second artificial intelligence model being configured to be pretrained to determine the compression garment fit information corresponding to the body part of the person based on the determined dimension information.

6. The method of claim 5, wherein the second artificial intelligence model is configured to be pretrained to determine the compression garment fit information corresponding to the body part of the person further based on additional information of the person.

7. The method of claim 6, wherein the second artificial intelligence model comprises different versions, each version being separately trained with data corresponding to a different targeted body part.

8. The method of claim 6, wherein the second artificial intelligence model comprises different versions, each version being separately trained with data corresponding to a different combination of a targeted body part and value ranges of parameters in the additional information.

9. The method of claim 1, wherein the compression garment fit information comprises tension values.

10. The method of claim 9, wherein each of the tension values is calculated based on a corresponding predetermined tension factor and corresponding skin surface dimension values.

11. The method of claim 9, wherein the tension values are determined by the second artificial intelligence model based on the determined dimension information and/or the additional information of the person, or the tension values are determined by a third artificial intelligence model based on the determined dimension information and/or the additional information of the person.

12. The method of claim 11, wherein the third artificial intelligence model comprises different versions, each version being separately trained with data corresponding to a different targeted body part, and/or corresponding to a different combination of a targeted body part and value ranges of parameters in the additional information.

13. The method of claim 5, wherein the second artificial intelligence model and/or the third artificial intelligence model comprises a version being trained to target a body shape that is asymmetric.

14. The method of claim 6, further comprising a step of receiving the additional information via user input.

15. The method of claim 6, wherein the additional information comprises at least one of height, age, weight, body mass index (BMI), and gender of the person.

16. The method of claim 1, wherein the compression garment fit information includes at least one of the circumferences to be measured according to RAL-GZ387/1 or RAL-GZ387/2, for example a waist circumference, an upper hip circumference, a calf B1 circumference and a foot Y circumference.

17. The method of claim 1, wherein the compression garment fit information comprises fit information for one or more prefabricated and/or preconfigured compression garments.

18. A computing device configured to perform the method of claim 1.

19. A storage medium configured to store instructions configured to be executed by at least one processor to perform the method of claim 1.
